(19) United States
(12) Patent Application Publication
(10) Pub. No.: US 2015/0258271 A1
(43) Pub. Date: Sep. 17, 2015

(54) METHODS OF ASSESSING INTRADUCTAL DISEASE

(71) Applicant: DR. SUSAN LOVE RESEARCH FOUNDATION, Santa Monica, CA (US)

(72) Inventor: Susan Love, Pacific Palisades, CA (US)

(21) Appl. No.: 14/523,123

(22) Filed: Oct. 24, 2014

Related U.S. Application Data
(60) Provisional application No. 61/895,951, filed on Oct. 25, 2013.

(51) Int. Cl. A61M 5/00; A61K 49/22; A61N 5/10; A61K 9/00

(52) U.S. Cl. CPC A61M 5/007; A61K 9/0041; A61K 49/22; A61N 5/10

(57) ABSTRACT

According to some embodiments, a method of evaluating an extent of intraductal disease in a breast of a subject comprises identifying a target duct of the breast of a subject, wherein the target duct comprises or is likely to comprise at least one abnormality, accessing the target duct through a corresponding ductal orifice of the breast using at least one access device, delivering a volume of a fluid within the target duct and imaging the breast using an imaging device to visualize and evaluate the extent of intraductal disease of the target duct.

### CROSS REFERENCE TO RELATED APPLICATIONS AND INCORPORATION BY REFERENCE

This application claims the priority benefit under 35 U.S.C. §119(e) of U.S. Provisional Application No. 61/895,951, filed Oct. 25, 2013, the entirety of which is hereby incorporated by reference herein. The disclosures of PCT Application No. PCT/US2008/063545, filed on May 13, 2008 and published as WO 2008/141318 on Nov. 20, 2008, U.S. patent application Ser. No. 11/291,738, filed on Dec. 1, 2005 and issued as U.S. Pat. No. 7,487,779 on Feb. 10, 2009, U.S. Provisional Patent No. 61/778,001, filed on Mar. 12, 2013, and U.S. patent application Ser. No. 14/204,848, filed on Mar. 11, 2014, are hereby incorporated herein in their entireties and made a part of the present application. Any and all priority claims identified in the Application Data Sheet, or any correction thereto, are hereby incorporated by reference under 37 CFR §1.57.

### BACKGROUND

1. Field

This application is directed to methods and systems for imaging and evaluating breast ducts.

2. Description of the Related Art

Noninvasive lesions in the breast (e.g., atypical hyperplasia, ductal carcinoma in situ or DCIS, lobular carcinoma, etc.) are being diagnosed at greater rates since the introduction of mammography screening and magnetic resonance imaging (MRI). Such lesions have the potential to evolve into invasive disease. However, the inability to distinguish which lesions are most likely to progress has led to the practice of aggressively treating all lesions with combinations of surgery, radiation and/or hormonal therapy. While these approaches have led to a demonstrated reduction in the local recurrence of noninvasive lesions and progression to invasive disease, they are associated with undesirable physical and emotional effects and unnecessary expense to the health care system. The anatomy of the ductal system of the breast makes it especially difficult to analyze and access (e.g., for treatment). The various embodiments disclosed herein are directed to improved methods and associated systems of identifying, imaging and evaluating targeted ducts of the breast for purposes of facilitating the treatment, motoring and/or other evaluation of a particular subject.

### SUMMARY

According to some embodiments, a method of evaluating an extent of intraductal disease in a breast of a subject comprises identifying a target duct of the breast of a subject, wherein the target duct comprises or is likely to comprise at least one abnormality, accessing the target duct through a corresponding ductal orifice of the breast using at least one access device, delivering a volume of a fluid within the target duct and imaging the breast using an imaging device to visualize and evaluate the extent of intraductal disease of the target duct.

According to some embodiments, the method further comprises initially identifying at least one abnormality within or near the breast of the subject using a screening procedure. In some embodiments, the screening procedure comprises a mammogram, x-ray, a MRI procedure or any other imaging procedure. In some embodiments, the at least one abnormality comprises ductal carcinoma in situ (DCIS) or a precancerous growth.

According to some embodiments, accessing the target duct comprises cannulating the target duct via the corresponding ductal orifice. In some embodiments, cannulating the target duct comprises inserting a microcatheter or other access device within the target duct. In some embodiments, the microcatheter comprises a galactogram catheter (e.g., 20-30 gauge, etc.), another type of intravenous catheter, a cannula, any other access device having a lumen or opening.

According to some embodiments, the fluid delivered into the target duct comprises a contrasting material. In some embodiments, the fluid delivered into the target duct comprises saline and/or a contrasting liquid or other fluid. In some embodiments, the fluid delivered into the target duct comprises a contrasting material.

According to some embodiments, the imaging device used to image the breast comprises an ultrasound device. In some embodiments, the ultrasound device comprises a 3D ultrasound device.

According to some embodiments, evaluating the extent of intraductal disease of the target duct comprises imaging the target breast over time to determine whether a size of the at least one abnormality has increased, decreased or remained the same. In some embodiments, evaluating the extent of intraductal disease of the target duct is performed in preparation for surgery or therapy of the subject. In some embodiments, surgery comprises at least partial resection of the area surrounding the at least one abnormality. In some embodiments, therapy comprises at least one of chemotherapy, radiation treatment or any other treatment intended to target the tissue at or near the at least one abnormality.

According to some embodiments, the methods and systems facilitate in determining whether or not a subject's condition has improved, worsened or stabilized (e.g., comparing various iterations of an evaluation to one another over time). For example, a subject can repeat a procedure periodically to determine whether, and to what extent, a disease is changing (e.g., DCIS is increasing or decreasing in size, spreading, etc.). Thus, in some embodiments, the methods, devices and systems disclosed herein are used to delineate or otherwise determine the extent of a disease, and not merely to diagnose or detect a disease.

### DETAILED DESCRIPTION

According to some embodiments, methods of identifying and evaluating breast ducts of subjects for assessing intraductal disease are disclosed. As discussed in greater detail herein, such methods can include the use of imaging technologies, such as, for example, ultrasound imaging (e.g., 3D ultrasound), CT (CAT) scans, other imaging modalities and/or the like. The methods and systems disclosed herein can be used to, among other things, evaluate the extent of intraductal disease of a subject, monitor subjects (e.g., before, during and/or after surgery and/or therapy), direct surgery and/or the like. Although the various embodiments disclosed herein are described in the context of the ducts of a breast, the corresponding devices, systems and methods can be applied to other parts of an anatomy that include one or more ducts, lumens or other passages that are vulnerable to intraductal or intraluminal disease, such as cancer or other abnormal formations, blockages and/or the like.

In some embodiments, the risk assessment methods disclosed herein can be used either before or after an initial screening or testing procedure. For example, the methods can be used on subjects who have undergone a core biopsy that resulted in a diagnosis of ductal carcinoma in situ (DCIS) of the breast, a screening MRI, x-ray and/or other imaging evaluation (e.g., mammogram). However, in some embodiments, any of various evaluation embodiments disclosed herein can be used as an initial screening procedure (e.g., with or without prior screening or testing).

According to some embodiments, a target duct of the subject's breast that contained (or is suspected of containing) DCIS or any other abnormality (e.g., microcalcification) can be identified. Such an identification step can be accomplished with the assistance of one or more ductal mapping techniques. For example, radiopaque mapping and/or any other technology can be used to identify the targeted duct. Such mapping or other technologies can precisely or approximately identify where along the ductal system (e.g., location relative to the nipple, depth relative to the subject skin, location relative to some anatomical landmark, etc.) of the breast a possible DCIS or other abnormality is located.

In some embodiments, the targeted duct is identified by accessing a ductal orifice of the breast that correlates to the corresponding ductal passage. For instance, central ducts of the breast can project directly or generally backwardly (e.g., at least partially in the direction of the chest wall of the subject). In some embodiments, peripheral ducts of the breast can extend radially away from the center of the breast. In some embodiments, the actual mapped intraductal system of the subject being evaluated is used to identify the target breast duct. However, in other arrangements, an approximation of the intraductal system of the subject's breast is used (e.g., based at least in part, for example, on a typical intraductal system) to approximate the location of the ductal orifice that is associated with the potentially diseased target duct.

Regardless of the exact methods of identifying the duct with the possible DCIS or other abnormality, once identified, such a target duct can optionally undergo additional evaluation procedures, as disclosed herein. For example, in some embodiments, the target duct can be cannulated or otherwise accessed. A microcatheter, needle and/or other device can be passed through the ductal orifice and into the target duct. Any other device, technique or method of accessing the target duct (e.g., for the subsequent passage of fluids therethrough) can be used. In some embodiments, the microcatheter or other access device is advanced into the target duct far enough so as to pass the lactiferous sinus thereby enabling the instillation of fluid into the duct containing the possible DCIS or other abnormality. In some embodiments, the microcatheter comprises a galactogram catheter (e.g., 20-30 gauge, etc.), another type of intravenous catheter, a cannula, any other access device having a lumen or opening, etc.

In some embodiments, once the target duct of the subject's breast has been cannulated or otherwise accessed, one or more fluids can be selectively delivered through the ductal orifice and into the duct. For example, the proximal end of the microcatheter of other access device that has been advanced into the target duct of the subject can be placed in fluid communication with a fluid delivery device (e.g., using a luer or other standard or non-standard coupling or connection). The delivery of such fluids to the target duct can be intermittent or continuous, and/or may be performed using a manual fluid delivery device (e.g., hand-operated syringe) or an automated fluid delivery device (e.g., an electromechanical pump, other device comprising a pump, a pneumatic device, etc.), as desired or required. In some embodiments, the delivery of fluid into the target duct can be performed at a relatively low, gentle flowrate (e.g., under laminar flow conditions), so as to at least partially hydrate or fill the duct but not disturb or otherwise impact the native anatomy of the duct and surrounding tissue.

In some embodiments, if it is unclear which duct includes the possible abnormality (e.g., DCIS), two or more (e.g., 3, 4, 5, 6, more than 6, etc.) ducts that are likely to include the possible abnormality can be accessed (e.g., cannulated) and injected with a fluid. Such a procedure (e.g., where more than one duct is accessed) can be performed as a matter of course, depending on the protocol. For example, if the prescreening methods or procedures that led to a diagnosis of possible intraductal disease were relatively crude or inaccurate, more than one duct (e.g., 2, 3, 4, 5, 6, more than 6, etc.) can be cannulated and treated with a fluid.

According to some embodiments, the fluid that is delivered to the target duct of the subject's breast comprises saline, an imaging (e.g., ultrasound, radiographic, MRI, etc.) contrast material and/or any other fluid. Depending on the protocol, once the desired or required volume of contrast or other fluid has been delivered into the target duct, imaging techniques can be used on the breast, as discussed in greater detail herein.

In some embodiments, an ultrasound device is used to image the targeted duct or ducts through which fluid (e.g., saline, other contrast liquid or material, etc.) was previously delivered. In some embodiments, the ultrasound device comprises a three-dimensional (3D) ultrasound. Such 3D imaging technologies can advantageously allow a user to more accurately visualize the targeted duct or ducts. However, standard ultrasound (e.g., 2D ultrasound) and/or other imaging technology can be used. In other embodiments, any other type of imaging device or technology can be used, such as, for example, magnetic resonance imaging (MRI), computed tomography (CT), other scans, x-ray and/or the like.

The imaging of the target duct or ducts of the subject's breast can be used to assess risk, determine the extent of disease, monitor subjects who are undergoing treatment (e.g., for their precancerous disease), facilitate preparation of a subsequent surgery (e.g., resection) and/or treatment procedure (e.g., post or pre-operative chemotherapy, radiation treatment, other therapy, etc.), evaluate the level of success following a procedure, document the status of a particular subject or associated procedure or treatment protocol and/or the like.

For example, in some embodiments, the intraductal evaluation and imaging embodiments disclosed herein can be used to determine whether a subject needs to undergo therapy and/or surgery in an effort to remove and/or reduce the size of a DCIS or other abnormality identified in one or more ducts of the subject's breast. Likewise, the embodiments disclosed herein can facilitate in determining whether or not a subject's condition has improved, worsened or stabilized (e.g., comparing various iterations of an evaluation to one another over time). For example, a subject can repeat a procedure periodically (e.g., weekly, monthly, bi-monthly, semi-annually, annually, etc.) to determine whether, and to what extent, a disease is changing (e.g., DCIS is increasing or decreasing in size, spreading, etc.). Thus, in some embodiments, the methods, devices and systems disclosed herein are used to delineate or otherwise determine the extent of a disease, and not merely to diagnose or detect a disease.

To assist in the description of the disclosed embodiments, words such as upward, upper, bottom, downward, lower, rear, front, vertical, horizontal, upstream, downstream have been used above to describe different embodiments and/or the accompanying figures. It will be appreciated, however, that the different embodiments, whether illustrated or not, can be located and oriented in a variety of desired positions (e.g., radially, longitudinally, transversely, diagonally, etc.).

Although several embodiments and examples are disclosed herein, the present application extends beyond the specifically disclosed embodiments to other alternative embodiments and/or uses of the inventions and modifications and equivalents thereof. It is also contemplated that various combinations or subcombinations of the specific features and aspects of the embodiments may be made and still fall within the scope of the inventions. Accordingly, it should be understood that various features and aspects of the disclosed embodiments can be combine with or substituted for one another in order to form varying modes of the disclosed inventions. Thus, it is intended that the scope of the present inventions herein disclosed should not be limited by the particular disclosed embodiments described above, but should be determined only by a fair reading of the claims that follow.

Various embodiments of the invention have been presented in a range format. It should be understood that the description in range format is merely for convenience and brevity and should not be construed as an inflexible limitation on the scope of the invention. The ranges disclosed herein encompass any and all overlap, sub-ranges, and combinations thereof, as well as individual numerical values within that range. For example, description of a range such as from 70 to 115 degrees should be considered to have specifically disclosed subranges such as from 70 to 80 degrees, from 70 to 100 degrees, from 70 to 110 degrees, from 80 to 100 degrees etc., as well as individual numbers within that range, for example, 70, 80, 90, 95, 100, 70.5, 90.5 and any whole and partial increments therebetween. Language such as “up to,” “at least,” “greater than,” “less than,” “between,” and the like includes the number recited. Numbers proceeded by a term such as “about” or “approximately” include the recited numbers. For example, “about 4 mm” includes “4 mm”.

While the inventions disclosed herein are susceptible to various modifications, and alternative forms, specific examples thereof have been shown in the drawings and are herein described in detail. It should be understood, however, that the inventions are not to be limited to the particular forms or methods disclosed, but to the contrary, the inventions are to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “moving a force applicator” include “instructing the movement of a force applicator.”

What is claimed is:

1. A method of evaluating an extent of intraductal disease in a breast of a subject, the method comprising:
identifying a target duct of the breast of a subject, wherein the target duct comprises or is likely to comprise at least one abnormality;
accessing the target duct through a corresponding ductal orifice of the breast using at least one access device;
delivering a volume of a fluid within the target duct; and
imaging the breast using an imaging device to visualize and evaluate the extent of intraductal disease of the target duct.

2. The method of claim 1, further comprising initially identifying at least one abnormality within or near the breast of the subject using a screening procedure.

3. The method of claim 2, wherein the screening procedure comprises a mammogram, a MRI procedure or any other imaging procedure.

4. The method of claim 1, wherein the at least one abnormality comprises ductal carcinoma in situ (DCIS) or a precancerous growth.

5. The method of claim 1, wherein accessing the target duct comprises cannulating the target duct via the corresponding ductal orifice.

6. The method of claim 5, wherein cannulating the target duct comprises inserting a microcatheter or other access device within the target duct.

7. The method of claim 1, wherein the fluid delivered into the target duct comprises a contrasting material.

8. The method of claim 1, wherein the fluid delivered into the target duct comprises saline and/or another contrasting liquid.

9. The method of claim 1, wherein the fluid delivered into the target duct comprises a contrasting material.

10. The method of claim 1, wherein the imaging device used to image the breast comprises an ultrasound device.

11. The method of claim 10, wherein the ultrasound device comprises a 3D ultrasound device.

12. The method of claim 1, wherein evaluating the extent of intraductal disease of the target duct comprises imaging the target breast over time to determine whether a size of the at least one abnormality has increased, decreased or remained the same.

13. The method of claim 12, wherein the evaluation is repeated periodically according to a set schedule.

14. The method of claim 13, wherein the evaluation is repeated weekly, monthly, semi-annually or annually.

15. The method of claim 1, wherein evaluating the extent of intraductal disease of the target duct is performed in preparation for surgery or therapy of the subject.

16. The method of claim 15, wherein surgery comprises at least partial resection of the area surrounding the at least one abnormality.

17. The method of claim 15, wherein therapy comprises at least one of chemotherapy, radiation treatment or any other treatment intended to target the tissue at or near the at least one abnormality.
